(19) United States
(12) Patent Application Publication
(10) Pub. No.: US 2005/0132431 A1
(43) Pub. Date: Jun. 16, 2005

(54) Method for synthesizing ectomycorrhiza in vitro

(75) Inventors: Andrew P. Coughlan, Quebec (CA); Yves Piche, Cap-Rouge (CA)

Correspondence Address: OGILVY RENAULT, 1981 MCGILL COLLEGE AVENUE, SUITE 1600, MONTREAL, QC, H3A2Y3, CA

(73) Assignee: UNIVERSITE LAVAL, Quebec (CA)

(21) Appl. No.: 10/813,070

(22) Filed: Mar. 31, 2004

Related U.S. Application Data
(60) Provisional application No. 60/459,995, filed on Apr. 4, 2003.

(51) Int. Cl. C12N 1/14; C12N 1/16; C12N 1/18; A01H 1/00; A01H 1/02

(52) U.S. Cl. 800/260

(57) ABSTRACT

The present invention relates to a method for colonizing a root-organ from a plant with a mycorrhizal fungus that is representative of the naturally process of colonization of plant roots by mycorrhizal fungi. The present invention also relates to the use of this method for studying in vitro the colonization of plant roots with a mycorrhizal fungus and to an in vitro model for studying the colonization of plant roots with a mycorrhizal fungus.

### BACKGROUND OF THE INVENTION

(a) Field of the Invention

The present invention relates to methods for colonizing root-organs from a plant with a mycorrhizal fungus and for studying in vitro the colonization of plant roots with a mycorrhizal fungus. The present invention also relates to the use of these methods and to an in vitro model for studying the colonization of plant roots with a mycorrhizal fungus.

(b) Description of Prior Art

Mycorrhiza are symbiotic associations in which fungi become integrated into the physical structure of the roots of a plant. Ectomycorrhiza (EM) and endomycorrhiza are the two basic types of mycorrhizal associations. Endomycorrhizal fungi invade the living cells of the root which become filled with mycelial clusters. In a widespread form of endomycorrhiza, the microscopic appearance of intracellular hyphal clusters leads to the name of vesicular-arbuscular (VA) mycorrhiza. By contrast, the EM fungal hyphae penetrate the intracellular spaces of the epidermis and of the cortical region of the root but do not invade the living cells. The morphology of the root is altered, forming a shorter, dichotomously branching cluster with a reduced meristematic region. The external pseudoparenchymatous sheath formed by EM fungi can constitute up to 40% of the dry weight of the combined root-fungus structure. Ectomycorrhizal associations are commonly found in gymnosperms (that include pine, spruce and fir) and angiosperm species (that include oak, beech and birch).

The number of plants capable of normal development in the absence of mycorrhizal involvement is very limited. The majority of plants rather rely on such mycorrhizal association for their normal growth and development. For example, EM act as an extension of the colonized plant's roots, increasing the plant's absorbative surface by approximately 700%, allowing additional uptake of water and nutrients. The EM association reduces drought stress and the need for artificial fertilizer and pesticides since the plant derives several benefits from its association with EM fungi, including increased longevity of feeder roots, increased rates of nutrient absorption from soil, selective absorption of certain ions from soil, resistance to plant pathogens, increased tolerance to toxins and increased tolerance to extremes of a range of environmental parameters, such as temperature, drought and pH.

Ectomycorrhizal fungi also find advantages from EM association. Indeed, EM fungi benefit from carbohydrates, amino acids and vitamins produced by the plant. The symbiotic association between a plant and an ectomycorrhizal fungus is essential for completion of the fungal life cycle.

The study of the EM fungus Tuber melanosporum has increased in recent years because they are responsible for the formation of black truffles, which are a valued gastronomic product. Since the symbiosis between this fungus and a host plant is critical for black truffles development, the many attempts to grow black truffles in a sterile medium, in the absence of a host plant, remain unsuccessful. Therefore, the only way to produce black truffles known in the prior art is to harvest T. melanosporum associated with the roots of a plant.

To enhance the production of black truffles, plant roots are inoculated with T. melanosporum and further grown in a soil. Inoculation of roots with T. melanosporum is nearly always carried out with suspensions of spores or specially gathered mycorrhizal roots. Ectomycorrhizal fungal inoculum for woody plants such as pines have been produced. For example, U.S. Pat. No. 4,327,181, to Litchfield et al., discloses a liquid culture of selected fungi for broadcast over forest soil. U.S. Pat. No. 5,178,642 to Janerette discloses a process for production of inocula for herbaceous plants.

U.S. Pat. No. 4,749,402 delivered to Garrett et al. describes a method for enhancing the development of mycorrhizal fungi in the root system of a plant. Particularly, this invention relates to a nutrient composition adapted for foliar fertilization of a plant to enhance the development of mycorrhiza on the root system. This composition comprises magnesium, boron, nitrogen, ammonium sulfate, phosphorous, potassium and sulfur and has a pH ranging from 5.5 to 6.5.

While some methods for synthesizing EM both in vitro and ex vitro exist, they require actively growing plants and may represent additional expenses related to the cost of land or growth chambers. Furthermore, soil based substrates used in the prior art do not allow in situ visualization of the EM symbiosis and growth pouches preclude in vitro observations and understanding of the mechanisms subjacent to plant/fungus communication and interactions.

To overcome the above-identified problems, the prior art reports studies on the endomycorrhizal association with root-organ cultures of host plants. The use of root-organs allowed important breakthroughs in fundamental and applied endomycorrhizal research. Furthermore, the colonized root-organs found use as valuable sources of fungal inoculum for experiments involving whole plants, similarly to excised root from whole non-transformed EM plants. However, root-organs of trees are extremely difficult to establish on artificial media and since most plants on which EM are found are trees, a severe obstacle to the use of root-organs in EM studies remains. To overcome it, the prior art reports the use of shrubby plants that form EM, such as Cistus. However, this technique remains imperfect since five months are required to achieve EM formation while EM formation under natural conditions takes only a few days. Therefore, the methods for the colonization of shrubby plant root-organs with EM fungi known in the prior art are not likely to be the most appropriate models to study EM associations.

Considering the state of the art, it would be highly desirable to be provided with a new and faster in vitro method for the production of a system based on the use of root-organ cultures, for the study of EM, and for the production of inocula for the inoculation of whole plants.

### SUMMARY OF THE INVENTION

An object of the present invention is to provide a method for colonizing root-organs from a plant with a mycorrhizal fungus. The method comprises:
obtaining a root-organ from a plant;
growing the root-organ in a first culture medium;
removing a portion of the first culture medium in the proximity of at least one developing lateral root of the root-organ;
replacing the removed portion of the first culture medium with a portion of a second culture medium, which has previously been inoculated with the mycorrhizal fungus; and
allowing the developing lateral root to grow through the portion of the second culture medium to contact the mycorrhizal fungus.

Another object of the present invention is to provide the use and an in vitro model of the above-described method to study in vitro the colonization of plant roots with an EM fungus.

### DESCRIPTION OF PREFERRED EMBODIMENTS

The present invention provides a method for colonizing root-organ from a plant with an EM fungus and a method for studying the colonization of plant roots by an EM fungus in vitro. For the purpose of the present invention, any fungus may be used to colonize the root-organ or the roots of a plant, but an EM fungus is preferably used, such as a fungus belonging to the genera Boletus, Rhizopogon, Tricholoma or Tuber. Tuber genus fungi are preferably used as inoculum and more preferably the species Tuber melanosporum, since it produces the black truffle, which is the most valuable EM fungal fruit body.

The root-organ used for the purpose of the present invention may be obtained from any plant known in the art to be capable of producing such organs. However, the root-organ is preferably obtained from a tree or a shrubby plant, more preferably from a Cistus and even more preferably from a Cistus incanus.

The first and the second culture medium of the present invention may have a similar composition and consistency, but are preferably identical culture media. These culture media may be liquid or semi-liquid media but solid culture media are preferred. Any skilled artisan would understand that any medium enabling the concomitant growth or propagation of a plant or of a plant tissue and of a fungus may be used for the purpose of the present invention and includes White's Medium and its derivatives such as modified whites (WM) medium. However, minimal (M) medium, as reported by Bécard and Fortin in 1988 is preferably used for the purpose of the present invention.

Minimal medium comprises, for one liter: 731 mg MgSO₄.7H₂O, 80 mg KNO₃, 65 mg KCl, 4.8 mg KH₂PO₄, 288 mg Ca(NO₃)₂.4H₂O, 8 mg NaFeEDTA, 0.75 mg Kl, 6 mg MnCl₂.4H₂O, 2.65 mg ZnSO₄7H₂O, 1.5 mg H₃BO₃, 0.13 mg CuSO₄.5H₂O, 0.0024 mg Na₂MoO₄.2H₂O, 3 mg C₂H₅NO₂ (Glycine), 0.1 mg C₁₂H₁₈Cl₂N₄OS (Thiamine hydrochloride), 0.1 mg C₈H₁₂ClNO₃ (Pyridoxine hydrochloride), 0.5 mg C₆H₅NO₂ (Nicotinic acid), 0.50 mg C₆H₁₂O₆ (Myo-inositol), 10 g Sucrose and 5.5 g Gel-Gro,. The pH of the medium can be adjusted with KOH to suit the optimal growth of the fungus species used therein and preferably ranges from 5.5 to 6.5.

To allow the root-organ of the present invention to contact the fungus, a portion of the first culture medium is removed, in the vicinity of at least one developing lateral root of the root-organ and is replaced by a portion of the second culture medium. This portion of a second culture medium is inoculated with the mycorrhizal fungus prior to substituting the portion of the first culture medium. The second culture medium may be inoculated with a fungus by any proper method, but is preferably inoculated using a fungus culture or an existing root-organ, itself colonized with a fungus. The portion of the first culture medium may be removed anywhere in the neighborhood of at least one lateral root of the root-organ that will allow the subsequent growth of that lateral root through the substituted and inoculated portion of culture medium. The portion of the first culture medium is preferably removed adjacently to the tip of the lateral root so that the lateral root will naturally grow longer through the substituted culture medium. The removed portion may have any shape but a cylindrical shape is preferred. The cylindrical gel plug removed may have a diameter that varies from 2 to 30 mm, but a culture medium plug having a diameter of 8 mm is preferred. The gel plug removed can be replaced by a gel plug of the second culture medium that has any shape and size, but a gel plug having substantially identical shape and size to the removed gel plug is preferred.

The methods of the present invention allows the colonization of the root-organ with the fungus within a period of time similar to the period of time required for a fungus to colonize plant roots in naturally occurring conditions. The colonization of root-organs with the mycorrhizal fungus according to the method of the present invention occurs within a month, and more preferably within a two-week period and even more preferably within a week from the substitution of the first culture medium plug by the plug from the inoculated culture medium.

The methods of the present invention are intended for colonizing root-organ from a plant with a mycorrhizal fungus and for studying in vitro the colonization of plant roots by a mycorrhizal fungus. A person skilled in the art will understand that colonization of a root-organ with a mycorrhizal fungus may initiate numerous naturally occurring phenomena that may alter the morphology of either the root-organ or fungus. For example, the symbiotic association between a fungus and a root is known in the art to stimulate spore germination and hyphal growth of the fungus. A skilled artisan will therefore understand that the present invention may serve the purpose of studying in vitro every aspect related to plant root colonization by mycorrhizal fungi.

The present invention will be more readily understood by referring to the following examples which are given to illustrate the invention rather than to limit its scope.

### EXAMPLE I

### Preparation of Cistus incanus Root-Organ Cultures

The plant from which a root-organ culture is obtained is preferably Cistus incanus, a shrubby plant known to form EM associations with fungi. Seeds of C. incanus were obtained from the Institut Botanique de I'Université Coimbra, Portugal (Universidade-Coimbra) and the Orto Botanico dell'Universita, Via P. A. Mattiolo n.4, 53100 Siena, Italy.

Briefly, axenic seedlings of C. incanus were obtained by germinating, in glass Petri dishes filled with damp sterilized sand, seeds that were surface sterilized with H₂O₂ (30 vols.) for 15 to 20 minutes and heat treated at 100° C. for 20 to 30 minutes.

Using a sterile syringe needle, seedlings were wounded on one of their leaves and were inoculated after 1 to 2 minutes with cells of A. rhizogenes sampled from a 48-hour-old culture. The A. rhizogenes isolate LBA 9402 used for the purpose of the present invention was supplied by Dr David Tepfer (Laboratoire de Biologie de la Rhizosphere, Institut National de la Recherche Agronomique (INRA), F-78026, CEDEX Versailles, France). Cultures of the LBA 9402 A. rhizogenes isolate were maintained on yeast-mannitol agar medium (pH 7) that comprises, for one liter of culture medium, 10 g mannitol, 0.4 g yeast extract, 0.1 g NaCl, 0.5 g K₂HPO₄, 2.0 g MgSO₄.7H₂O and 15 g agar. Inoculated seedlings were incubated under ambient conditions for a 2 week period, after which transformed roots were obtained at the wound sites.

To remove A. rhizogenes from the transformed roots and to develop root-organ cultures, root tips (2-3 cm) were transferred to a modified White's medium (WM) amended with either rifampicin (50 mg/L) or a mix of cefotaxime 200 mg/L and carbenicillin 500 mg/L. The WM medium comprises, for one liter of culture medium, 731 mg MgSO₄.7H₂O, 453 mg Na₂SO₄.10H₂O, 80 mg KNO₃, 65 mg KCl, 21.5 mg NaH₂PO₄.2H₂O, 288 mg Ca(NO₃)₂.4H₂O, 8 mg NaFeEDTA, 0.75 mg Kl, 6 mg MnCl₂4H₂O, 2.65 mg ZnSO₄.7H₂O, 1.5 mg H₃BO₃, 0.13 mg CuSO₄.5H₂O, 0.0024 mg Na₂MoO₄.2H₂O, 3 mg C₂H₅NO₂ (Glycine), 0.1 mg C₁₂H₁₈Cl₂N₄OS (Thiamine hydrochloride), 0.1 mg C₈H₁₂ClNO₃ (Pyridoxine hydrochloride), 0.5 mg C₆H₅NO₂ (Nicotinic acid), 50 mg C₆H₁₂O₆ (Myo-inositol), 30 g sucrose and 3.5 g Gel-Gro.

The pH was adjusted to 6.5 (using KOH) before adding the solution to the gelling agent. Once a week, actively growing root tips were transferred to fresh WM medium with antibiotics to obtain bacteria-free root-organs after four or five successive transfers. The bacteria-free Cistus root-organ cultures were maintained on WM medium pH 6.5, in 150 mm Petri dishes and incubated in the dark at 25° C., and 2 cm-long apical tips were transferred to fresh media once every 14 days.

Five root-organ clones from C. incanus were obtained with the method described herein above, namely clones 1, 1B, 2, 3 and 4. Since clone #2 was shown to be the most vigorous, it was selected for subsequent experiments.

### EXAMPLE II

### Colonization of Root-Organs with an Ectomycorrhizal Fungus

The colonization of root-organs with a selected fungus was performed by transferring 2 cm-long root tip segments from an actively growing C. incanus root-organ culture (clone #2) into a 150 mm Petri dish comprising fresh WM medium and by incubating it for seven days. The product of this incubation was then transferred into a recipient containing minimal (M) medium. Minimal medium comprises, for one lifer: 731 mg MgSO₄.7H₂O, 80 mg KNO₃, 65 mg KCl, 4.8 mg KH₂PO₄, 288 mg Ca(NO₃)₂.4H₂O, 8 mg NaFeEDTA, 0.75 mg Kl, 6 mg MnCl₂.4H₂O, 2.65 mg ZnSO₄.7H₂O, 1.5 mg H₃BO₃, 0.13 mg CuSO₄.5H₂O, 0.0024 mg Na₂MoO₄.2H₂O, 3 mg C₂H₅NO₂ (glycine), 0.1 mg C₁₂H₁₈Cl₂N₄OS (thiamine hydrochloride), 0.1 mg C₈H₁₂ClNO₃ (pyridoxine hydrochloride), 0.5 mg C₆H₅NO₂ (nicotinic acid), 50 mg C₆H₁₂O₆ (Myo-inositol), 10 g sucrose and 5.5 g Gel-Gro. The pH of the medium was adjusted with KOH to the optimal growth pH of the fungus species used therein and ranged from 5.5 to 6.5.

A gel plug adjoining the growing tip of a developing lateral root was removed and further replaced by an identical sized and shaped sample cut from a gel comprising an actively growing fungal colony. A wide range of identified and non-identified ectomycorrhizal fungi were tested, and more particularly T. melanosporum. Since the TMEL0199 strain was the most vigorous strain of T. melanosporum, it was elected for the purpose of the root-organ colonization. The root-organ was then enabled to grow through the fungus colony, becoming itself colonized by the ectomycorrhizal fungus.

Results

The EM formation occurred five days after root-hyphal contact between C. incanus clone #2 and T. melanosporum TMEL0199. Clones 1, 1 B, 3 and 4 also formed mycorrhiza with the tested fungi, within two to three weeks. The formation of EM with all isolates of known mycorrhizal fungi tested was obtained and root-organ cultures were used to stimulate growth and for the general maintenance of all cultures of Tuber species.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method for colonizing a root-organ from a plant with a mycorrhizal fungus that comprises:
obtaining said root-organ from said plant;
growing said root-organ in a first culture medium;
removing a portion of said first culture medium in the proximity of at least one developing lateral root of said root-organ;
replacing said removed portion of said first culture medium with a portion of a second culture medium, wherein said second culture medium has been previously inoculated with said mycorrhizal fungus; and
allowing said at least one developing lateral root to grow through said portion of a second culture medium to contact said mycorrhizal fungus.

2. The method of claim 1, wherein said mycorrhizal fungus is an ectomycorrhizal fungus.

3. The method of claim 2, wherein said ectomycorrhizal fungus is Tuber melanosporum.

4. The method of claim 1, wherein said plant is a tree or a shrubby plant.

5. The method of claim 4, wherein said shrubby plant is a Cistus.

6. The method of claim 5, wherein said Cistus is Cistus incanus.

7. The method of claim 1, wherein said first culture medium and said second culture medium are solid culture media.

8. The method of claim 7, wherein said solid culture medium is minimal medium.

9. The method of claim 1, wherein said removed portion is a gel plug.

10. The method of claim 9, wherein said gel plug is a 8 mm diameter gel plug.

11. The method of claim 1, wherein said portion of said second culture medium is a gel plug.

12. The method of claim 11, wherein said portion of said second culture medium has shape and size similar to said removed portion.

13. Use of the methods of claim 1 to study in vitro the colonization of plant roots by a mycorrhizal fungus.

14. An in vitro model to study the colonization of plant roots by a mycorrhizal fungus, said model being obtained by the method according to claim 1.
